(19) United States
(12) Patent Application Publication
(10) Pub. No.: US 2018/0200104 A1
(43) Pub. Date: Jul. 19, 2018

(54) LONG-ACTING ICE PACK

(71) Applicant: YING-HSUAN LU, Brea, CA (US)

(72) Inventor: YING-HSUAN LU, Brea, CA (US)

(21) Appl. No.: 15/406,929

(22) Filed: Jan. 16, 2017

(51) Int. Cl. A61F 7/10; A61F 7/02

(52) U.S. Cl. CPC A61F 7/10; A61F 7/02; A61F 2007/0257; A61F 2007/0219; A61F 2007/0206; A61F 2007/108

(57) ABSTRACT

A long-acting ice pack includes a mixture of a cold preservation substance and a gel therein. The gel is mixed with a plurality of heat-insulating particles. For a portion of the ice pack, the heat-insulating particles can be used to block the transmission of the interior temperature of the ice pack and the outside temperature so as to decrease the area and efficiency of temperature transmission. The ice pack not only effectively extends the time of cold preservation, meeting the demand of ice compress or cold preservation for a long time, but also provides the most comfortable temperature for ice compress, without using a towel to cover the ice pack.

### FIELD OF THE INVENTION

The present invention relates to a long-acting ice pack, and more particularly to a long-acting ice pack able to extend the time of cold preservation and provide an appropriate temperature for ice compress.

### BACKGROUND OF THE INVENTION

In many situations of our daily lives, there is a need for ice compress. Ice compress is one of cryotherapy, which is simple to practice, easy to obtain an ice pack, and has a good curative effect, etc. Therefore, within 24 hours after an acute injury, people often use an ice pack to reduce pain, inhibit an inflammation, contract blood vessels, reduce edema, reduce the metabolism, and achieve the purpose of the treatment of acute sports injuries.

In the early conventional ice compress method, ices are put in a plastic bag, and the ice bag is directly in contact with the user's skin for ice compress. In this way, the ices melt fast, so the effect of the ice bag is not good. An ice pack 1 is developed as shown in FIG. 1. A bag body 12 is prefilled with a gel 11. The gel 11 has the function of cold preservation. The ice pack 1 is first frozen. When in use, the ice pack 1 is taken out from a freezer, and it provides the function of cold preservation for a period of time. However, the conventional ice pack 1 is often too cold when it is taken out from the freezer, so it is always covered with a towel for ice compress to avoid frostbite. Moreover, the temperature of the conventional ice pack 1 will rise very fast after temperature exchange when the ice pack 1 is in contact with the human body due to the outside temperature and the temperature of the human body, so the ice pack will lose the function of ice compress in a short time.

Accordingly, the inventor of the present invention has devoted himself based on his many years of practical experiences to solve these problems.

### SUMMARY OF THE INVENTION

The primary object of the present invention is to overcome the shortcomings of the prior art and to provide an ice pack. When in use, the initial temperature of the ice pack is appropriate, not too low, and the time of cold preservation is longer than the prior art. The ice pack can be used for ice compress for a long time, and its temperature is quite suitable for the human body.

In order to achieve the aforesaid object, the long-acting ice pack of the present invention comprises a mixture of a cold preservation substance and a gel therein. The gel is mixed with a plurality of heat-insulating particles. The heat-insulating particles can be used to block the transmission of the interior temperature of the ice pack and the outside temperature for a portion of the ice pack so as to decrease the area and efficiency of temperature transmission. The ice pack not only effectively extends the time of cold preservation, meeting the demand of ice compress or cold preservation for a long time, but also provides the most comfortable temperature for ice compress, without using a towel to cover the ice pack.

The ice pack comprises a body. The body comprises a cold preservation substance and a gel therein. The cold preservation substance may be one of methylcellulose and acrylic acid polymer, but not limited thereto. The gel may be a high water-absorbing molecular polymer, but not limited thereto. The cold preservation substance and the gel are disposed in a waterproof layer. The waterproof layer is covered with an outer covering layer. The feature of the present invention is that the gel is further mixed with a plurality of heat-insulating particles. The heat-insulating particles may be made of Styrofoam material and have a block shape, a strip shape, a sheet shape, or a spherical shape.

The mixing ratio of the heat-insulating particles can be increased or decreased as necessary. If the ratio is increased, the portion of the ice pack to block the transmission of the interior temperature of the ice pack and the outside temperature will be increased. On the contrary, if the mixing ratio of the heat-insulating particles is reduced, the portion of the ice pack to block the transmission of the interior temperature of the ice pack and the outside temperature will be decreased.

### BRIEF DESCRIPTION OF THE DRAWINGS

FIG. 1 is a structural schematic view of a conventional ice pack;

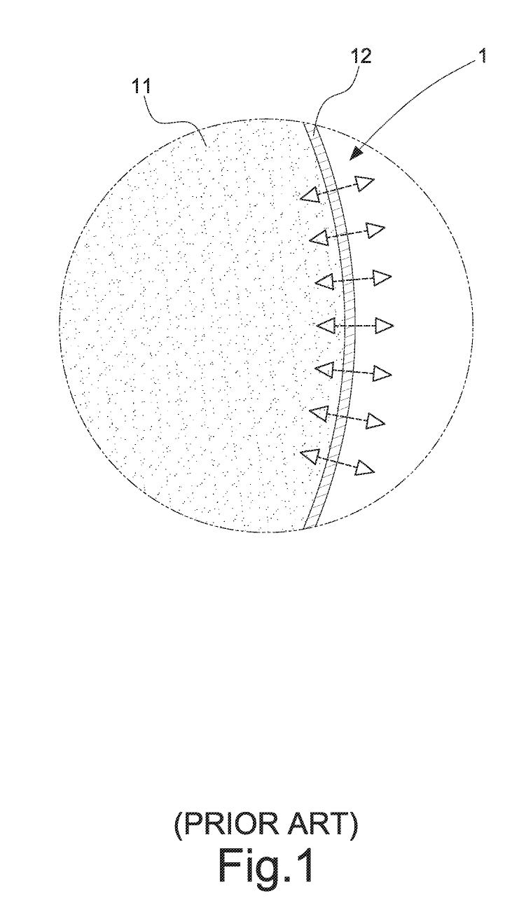

FIG. 2 is a structural schematic view of the present invention;

FIG. 3 is a schematic view of the present invention, showing the heat-insulating particles used to block the transmission of the interior temperature of the ice pack and the outside temperature for a portion of the ice pack; and

FIG. 4 is a schematic view showing the temperature and time curves of a comparison between the conventional ice pack and the ice pack of the present invention.

### DESCRIPTION OF THE PREFERRED EMBODIMENTS

In order to understand the present invention, embodiments of the present invention will now be described, by way of example only, with reference to the accompanying drawings.

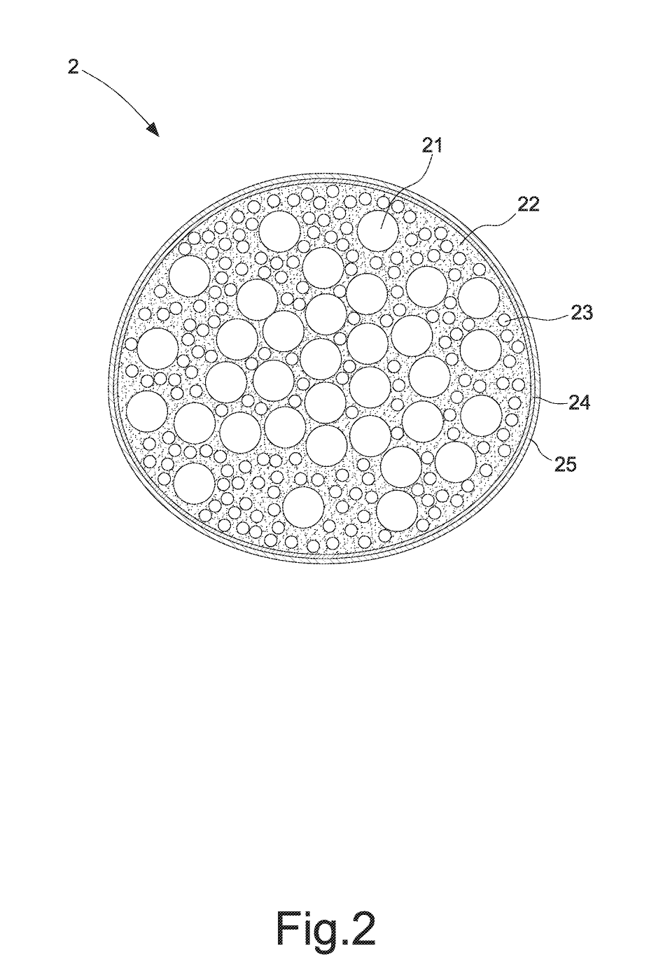

As shown in FIG. 2, the present invention discloses a long-acting ice pack. An ice pack 2 comprises a mixture of a cold preservation substance 21 and a gel 22 therein. The cold preservation substance 21 may be one of methylcellulose and acrylic acid polymer, but not limited thereto. The gel 22 may be a high water-absorbing molecular polymer, but not limited thereto. The cold preservation substance 21 and the gel 22 are disposed in a waterproof layer 24. The waterproof layer 24 is covered with an outer covering layer 25. The present invention is characterized in that the gel 22 is further mixed with a plurality of heat-insulating particles 23. The heat-insulating particles 23 may be made of Styrofoam material, but not limited thereto.

The mixing ratio of the heat-insulating particles 23 can be increased or decreased as necessary. If the ratio is increased, the portion of the ice pack for blocking the transmission of the interior temperature of the ice pack and the outside temperature will be increased. That is to say, the interior and exterior temperatures of the ice pack won't exchange easily. For the ice pack 2, the time of cold preservation time is prolonged, but the temperature sensed by the human body is relatively high during ice compress. On the contrary, if the mixing ratio of the heat-insulating particles 23 is reduced, the portion of the ice pack for blocking the transmission of the interior temperature of the ice pack and the outside temperature will be decreased. That is to say, the interior and exterior temperatures of the ice pack will exchange easily. For the ice pack 2, the time of cold preservation time will be shortened, but the temperature sensed by the human body will be relatively low during ice compress.

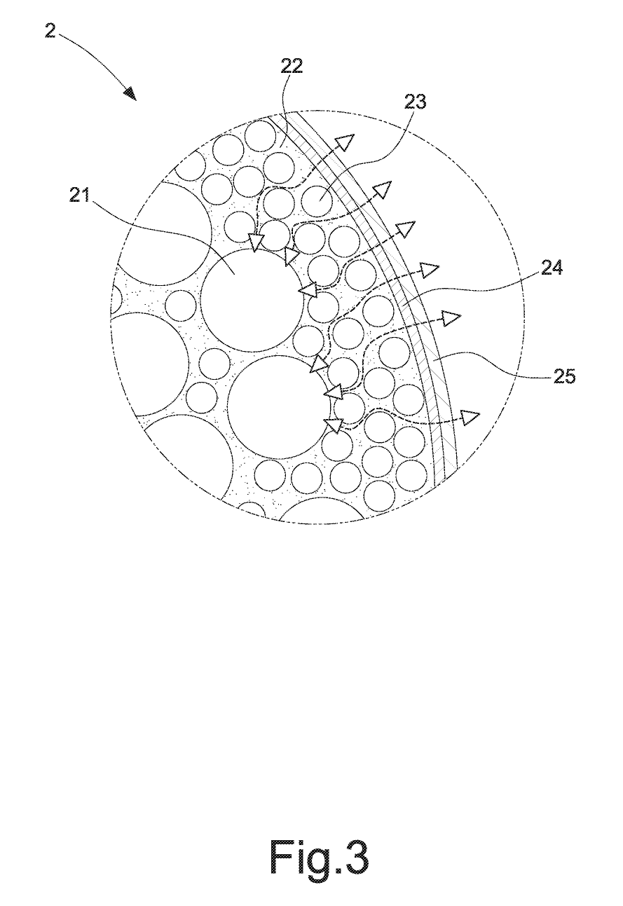

As shown in FIG. 3, to practice the present invention, the ice pack 2 is first put into a freezer and frozen for a period of time. The mixture of the cold preservation substance 21 and the gel 22 will be frozen. When the ice pack 2 is taken out from the freezer for use, the low temperature of the cold preservation substance 21 is blocked by the heat-insulating particles 23 so that the area of temperature exchange with the outside is greatly reduced. The temperature exchange is performed via the gap between the heat-insulating particles 23 or the gel 22. Thus, the ice pack of the present invention is able to provide an appropriate temperature for ice compress at the initial stage of use, without using a towel to cover the ice pack 2. Moreover, the heat-insulating particles 23 are configured to reduce the area of temperature exchange, such that the temperature of the ice pack 2 can be kept for a long time effectively.

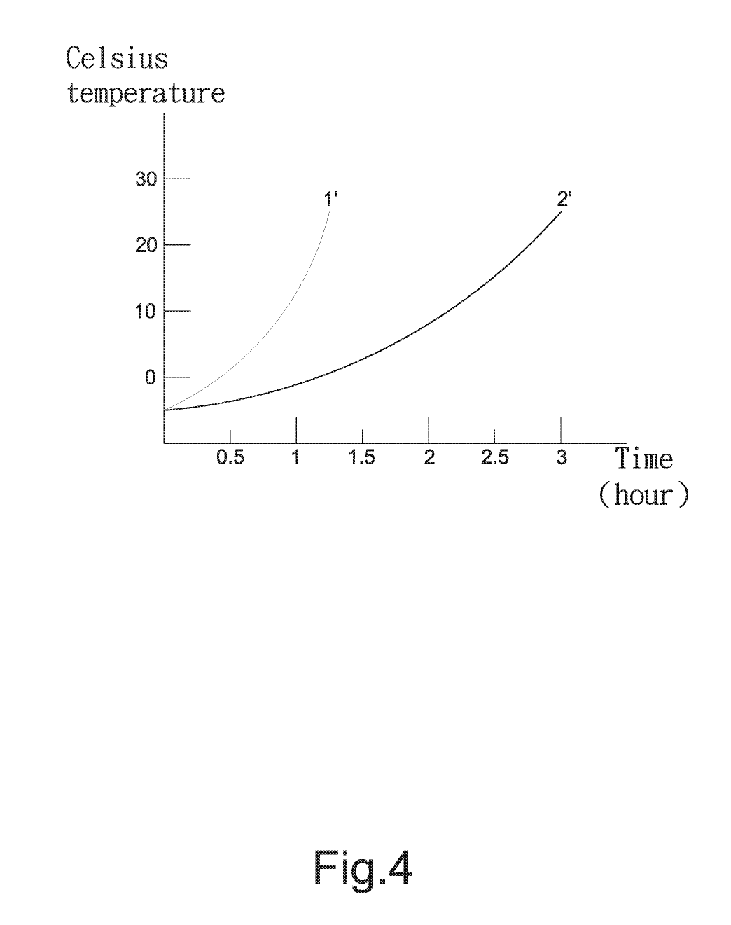

FIG. 4 is a schematic view showing the temperature and time curves of a comparison between the conventional ice pack 1 and the ice pack 2 of the present invention. The entire area of the conventional ice pack 1 can perform temperature exchange, so its temperature rises fast with time (curve 1′). On the contrary, the ice pack 2 of the present invention has the heat-insulating particles 23 to reduce the area of temperature exchange, which can preserve cold effectively. Thus, the time of cold preservation can be extended effectively (curve 2′).

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A long-acting ice pack, comprising a mixture of a cold preservation substance and a gel therein, and characterized in that the gel is mixed with a plurality of heat-insulating particles.

2. The long-acting ice pack as claimed in claim 1, wherein the cold preservation substance is one of methylcellulose and acrylic acid polymer.

3. The long-acting ice pack as claimed in claim 1, wherein the gel is a high water-absorbing molecular polymer.

4. The long-acting ice pack as claimed in claim 2, wherein the gel is a high water-absorbing molecular polymer.

5. The long-acting ice pack as claimed in claim 1, wherein the cold preservation substance and the gel are disposed in a waterproof layer, and the waterproof layer is covered with an outer covering layer.

6. The long-acting ice pack as claimed in claim 1, wherein the heat-insulating particles are made of Styrofoam material.

7. The long-acting ice pack as claimed in claim 1, wherein the heat-insulating particles each have one of a block shape, a strip shape, a sheet shape, and a spherical shape.
